(19) United States
(12) Patent Application Publication
(10) Pub. No.: US 2023/0372024 A1
(43) Pub. Date: Nov. 23, 2023

(54) SYNTHETIC POSITION IN SPACE OF AN ENDOLUMINAL INSTRUMENT

(71) Applicant: Covidien LP, Mansfield, MA (US)

(72) Inventor: Scott E.M. Frushour, Lafayette, CO (US)

(21) Appl. No.: 18/030,269

(22) PCT Filed: Nov. 2, 2021
(86) PCT No.: PCT/US2021/057764

§ 371 Date: Apr. 4, 2023

Related U.S. Application Data
(60) Provisional application No. 63/110,268, filed on Nov. 5, 2020.

(51) Int. Cl. A61B 34/20; A61B 1/313; A61B 1/018; A61B 5/06; G06T 7/62; G06T 7/70

(52) U.S. Cl. CPC A61B 34/20; A61B 1/3137; A61B 1/018; A61B 5/067; G06T 7/62; G06T 7/70; A61B 2034/2051; A61B 2034/2063; A61B 2034/2061; G06T 2207/10121; G06T 2207/10132; G06T 2207/10068; G06T 2207/30021

(57) ABSTRACT

A system and method of assessing a depth of view of an image by analyzing an image data set to determine a diameter of a luminal network proximate a determined position of a tool and displaying an image, the image including an indicator of a relative position of the catheter and the tool and an indicator of a position of a distal portion of the tool relative to a luminal wall of the luminal network.

### INTRODUCTION

This disclosure relates to surgical systems, and more particularly, to systems for intraluminal navigation and imaging with depth of view and distance determination.

### BACKGROUND

Knowledge of surgical tool location in relation to the internal anatomy is important to successful completion of minimally invasive diagnostic and surgical procedures. An endoscope or bronchoscope is the simplest form of navigation where a camera is placed at the distal tip of a catheter and is used to view the anatomy of the patient. Typically, the clinician uses their anatomic knowledge to recognize the current location of the bronchoscope. Near complex anatomic structures the clinician may attempt to analyze pre-surgical and intraprocedural patient images derived from any of computed tomography (CT) including cone beam CT, magnetic resonance imaging (MRI), positron emissions tomography (PET), fluoroscopy, or ultrasound scans to determine the location of the endoscope or tool associated therewith. For many luminal and robotic approaches stereoscopic imaging is either needed or beneficial to provide an adequate field of view (FOV) and an understanding of the depth of view (DOV) for the accurate placement of tools such as biopsy devices and ablation tools.

However, not all portions of the anatomy are amenable to the use of a two camera (stereoscopic) solution. In many instances the use of a second camera requires too much space and limits the ability for to use additional tools. These challenges can be particularly acute in the confined luminal spaces of the lung, esophagus, biliary ducts, and the urinary tract, but is also applicable to what are considered the relatively large lumen of the intestines and colon. Thus, improvements are needed to enable real time depth of view determinations to be presented without requiring the use of two cameras to produce stereoscopic views.

### SUMMARY

One aspect of the disclosure is directed to a method of assessing a depth of view of an image including: determining a position of a catheter in a luminal network, determining a position of a tool relative the catheter in the luminal network, acquiring an image data set. The method also includes analyzing the image data set to determine a diameter of the luminal network proximate the determined position of the tool; displaying an image acquired by an optical sensor secured to the catheter. The method also includes displaying an indicator of a relative position of the catheter and tool in the image acquired by the optical sensor and an indicator of a position of a distal portion of the tool relative to a luminal wall of the luminal network. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method further including displaying a distance of a closest point of the distal portion of the tool relative to the luminal wall. The method where the indicator includes at least two orthogonally measured distances of the distal portion of the tool relative to the luminal wall. The method where the image data set is a pre-procedure image data set. The method where the image data set is an intraprocedure image data set. The method where the position of the catheter is determined from data received from a sensor located in the catheter. The method where the position of the tool is determined from data received from a sensor located in the tool. The method where the sensor located in the catheter and in the tool are electromagnetic sensors. The method where the sensor located in the catheter and in the tool are inertial measurement units. The method where the sensor located in the catheter and in the tool are shape sensors. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A further aspect of the disclosure is directed to a system for depicting a depth of view (DOV) in an image including: a catheter including a first sensor configured for navigation in a luminal network and an optical sensor for generating an image; a tool including a second sensor configured to pass through a working channel in the catheter; a locating module configured to detect a position of the catheter and the tool; and an application stored on a computer readable memory and configured, when executed by a processor to execute the steps of. The system also includes registering data received from the first or second sensor with an image data set; analyzing an image data set to determine a diameter of a luminal network proximate the second sensor, and displaying the image generated by the optical sensor in combination with an indicator of a relative position of the catheter and tool and an indicator of a position of a distal portion of the tool relative to a luminal wall of the luminal network. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The system where the application executes a step of displaying distance of a closest point of the distal portion of the tool relative to the luminal wall. The system where the application executes a step of displaying at least two orthogonally measured distances of the distal portion of the tool relative to the luminal wall. The system where the image data set is a pre-procedure image data set. The system where the image data set is an intraprocedure image data set. The system where the intraprocedure image data set is received from a fluoroscope. The system where the sensor located in the catheter and in the tool are electromagnetic sensors. The system where the sensor located in the catheter and in the tool are inertial measurement units. The system where the sensor located in the catheter and in the tool are shape sensors. The system where the sensor located in the catheter and in the tool are ultrasound sensors. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

Various aspects and features of the disclosure are described hereinbelow with references to the drawings, wherein:

FIG. 1 is a schematic illustration of a system in accordance with the disclosure;

FIG. 2 is a schematic illustration of a distal portion of an endoscope or catheter with a tool passed therethrough in accordance with the disclosure;

FIG. 3 is an illustration of user interface in accordance with the disclosure; and

FIG. 4 is a flow chart detailing a method in accordance with the disclosure.

### DETAILED DESCRIPTION

This disclosure is directed to systems and methods for navigating within a luminal network and determining the distance a tool, observed in a field of view is from the camera. The disclosure is also directed to determining the distance of the tool from the luminal walls in which the tool in being navigated. In one embodiment the system and method use data derived from sensors placed on the endoscope and the tools to measure the relative distance between them. Additionally, image processing of the luminal network can be conducted of pre-procedure or intra-procedure images, and the detected positions of the endoscope and the tools determined relative to the images. The diameter of the luminal network and the position of the endoscope or the tools relative to the boundary walls of the luminal network determined and displayed in a live image from the endoscope. This depiction of relative distances of elements within a FOV enable assessment of depth of view (DOV) of an image and of the tools and structures found therein. These and other aspects of the disclosure are described in greater detail below.

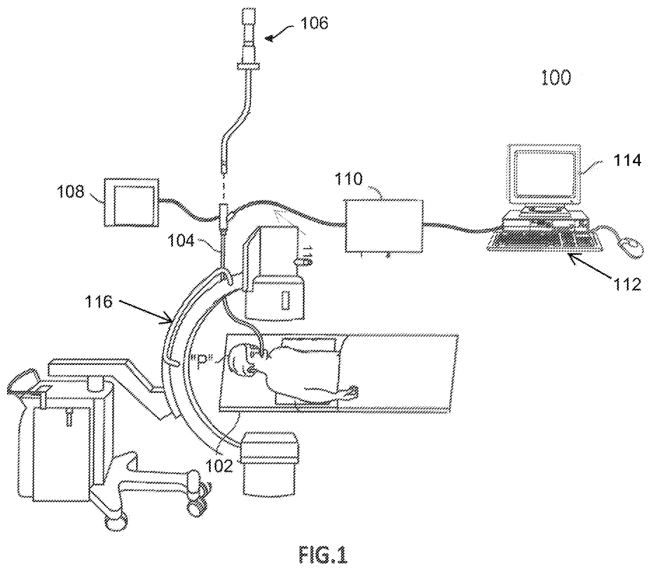

FIG. 1 is a perspective view of an exemplary system 100 in accordance with the disclosure. System 100 includes a table 102 on which a patient P is placed. A catheter 104 is inserted into an opening in the patient. The opening could be a natural opening such as the mouth, nose, or anus. Alternatively, the opening may be formed in the patient, for example a surgical port or a simple incision. The catheter 104 may be a bronchoscope including one or more optical sensors for capturing live images and video as the catheter 104 is navigated into the patient P. One or more tools 106, such as a biopsy needle, ablation needle, clamp forceps, or others may be inserted into the catheter 104 for diagnostic or therapeutic purposes. A monitor 108 may be employed to display images captured by the optical sensor on the catheter 104 as it is navigated within the patient P.

The system 100 includes a locating module 110 which receives signals from the catheter 104, and processes the signals to generate useable data, as described in greater detail below. A computer 112, including a display 114 receives the useable data from the locating module 110, and incorporates the data into one or more applications running on the computer 112 to generate one or more user-interfaces that are presented on the display 114. Both the locating module 110 and the monitor 108 may be incorporated into or replaced by applications running on the computer 112 and images presented via a user interface on the display 114. Also depicted in FIG. 1 is a fluoroscope 116 which may be employed in one or more methods as described in greater detail below to construct fluoroscopic based three-dimensional volumetric data of a target area from 2D fluoroscopic images and other imaging techniques. As will be appreciated the computer 112 incudes a computer readable recording medium such as a memory for storing image data and applications that can be executed by a processor in accordance with the disclosure to perform some or all of the steps of the methods described herein.

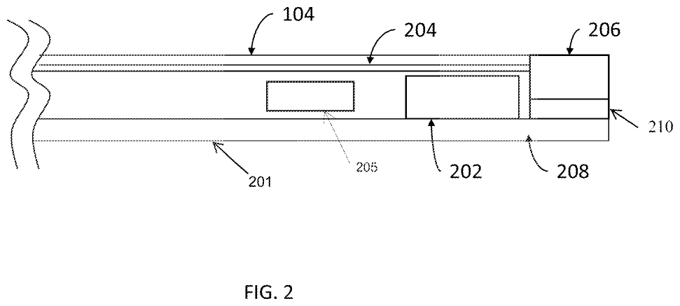

FIG. 2 depicts a further aspect of the disclosure related to the sensors that may be employed in connection with the catheter 104. In FIG. 2, the distal portion of the catheter 104 is depicted. The catheter 104 includes an outer sheath 201. A variety of sensors may be included in the distal portion of the catheter 104 including an inertial monitoring unit (IMU) 202, a shape sensor 204, an electromagnetic (EM) sensor 205 and an optical sensor 206 (e.g., a camera). In additional ultrasound sensors such as endobronchial ultrasound (EBUS) or radial endobronchial ultrasound (REBUS) may be employed. In one embodiment, one or more EBUS or REBUS sensors 210 may be placed proximate the distal portion of the catheter 104. In one embodiment they are placed in a distal face of the catheter 104 Though FIG. 2 multiple sensors installed in catheter 104, not all of the sensors are required in the systems or for performance of the methods of the disclosure. All that is required is that at least one such sensor output data which can be used to identify the location of the sensor and catheter 104 in the patient. Also shown in FIG. 2 is a working channel 208 through which one or more tools 106 may pass to acquire a biopsy, perform an ablation, or perform another medical function, as required for diagnosis and therapy. Each tool 106 also includes a sensor such as an IMU, EM sensor, shape sensor, optical sensor, etc. from which the position of the tool 106 can be determined by the locating module 110.

As shown in FIG. 2, the shape sensor 204, which may be an optic fiber such as a Fiber-Bragg grating, may connect with and be integrated into the optical sensor 206, such that the same optical fiber which carries the light captured by the optical sensor 206 is also utilized for shape sensing. The optical fiber forming the shape sensor 204 may be a single or a multi-core fiber as is known to those of ordinary skill in the art. As will be described in greater detail below, the IMU 202, shape sensor 204, EM sensor 205, optical sensor 206, or ultrasound sensor 210 are used to determine the location of the catheter 104 within the patient.

A further aspect of the disclosure is related to the use of linear EBUS and REBUS ultrasound sensors 210 described briefly above. In accordance with the ultrasound aspects of the disclosure a liner EBUS sensor may be placed in the distal face of the catheter 104. The result are forward looking ultrasound images can be acquired as the catheter 104 is navigated towards the target. Additionally or alternatively, the ultrasound sensors 210 are REBUS sensors, a 360-degree surrounding view of the distal portion of the catheter 104 can be imaged. Whether REBUS or EBUS, the sensors 210 can be used much like optical sensors to identify fiducials. Further, the images generated by the ultrasound sensors 210 can be compared to virtual ultrasound images generated from pre-procedure CT or MRI images to assist in confirming the location of the ultrasound sensor 210 (and catheter 104 therewith) while navigating towards the target.

There are known in the art a variety of pathway planning applications for pre-operatively planning a path through a luminal network such as the lungs or the vascular system. Typically, a pre-operative image data set such as one acquired from a CT scan or an MRI scan is presented to a user. The target identification may be automatic, semi-automatic, or manually, and allows for determining a pathway through patient P's airways to tissue located at and around the target. In one variation the user scrolls through the image data set, which is presented as a series of slices of the 3D image data set output from the CT scan. By scrolling through the images, the user manually identifies targets within the image data set. The slices of the 3D image data set are often presented along the three axes of the patient (e.g., axial, sagittal, and coronal) allowing for simultaneous viewing of the same portion of the 3D image data set in three separate 2D images.

Additionally, the 3D image data set (e.g., acquired from the CT scan) may be processed and assembled into a three-dimensional CT volume, which is then utilized to generate a 3D model of patient P's airways by various segmentation and other image processing techniques. Both the 2D slices images and the 3D model may be displayed on a display 114 associated with computer 112. Using computer 112, various views of the 3D or enhanced 2D images may be generated and presented. The enhanced two-dimensional images may possess some three-dimensional capabilities because they are generated from the 3D image data set. The 3D model may be presented to the user from an external perspective view, an internal “fly-through” view, or other views. After identification of a target, the application may automatically generate a pathway to the target. In the example of lung navigation, the pathway may extend from the target to the trachea, for example. The application may either automatically identify the nearest airway to the target and generate the pathway, or the application may request the user identify the nearest or desired proximal airway in which to start the pathway generation to the trachea. Once selected, the pathway plan, three-dimensional model, and 3D image data set and any images derived therefrom, can be saved into memory on the computer 112 and made available for use in combination with the catheter 104 during a procedure, which may occur immediately following the planning or at a later date.

Still further, without departing from the scope of the disclosure, the user may utilize an application running on the computer 112 to review pre-operative 3D image data set or 3D models derived therefrom to identify fiducials in the pre-operative images or models. The fiducials are elements of the patient's physiology that are easily identifiable and distinguishable from related features, and of the type that could typically also be identified by the clinician when reviewing images produced by the optic sensor 206 during a procedure. As will be appreciated these fiducials should lay along the pathway through the airways to the target. The identified fiducials, the target identification, and/or the pathway are reviewable on computer 112 prior to ever starting a procedure.

Though generally described herein as being formed pre-operatively, the 3D model, 3D image data set and 2D images may also be acquired in real time during a procedure. For example, such images may be acquired by a cone beam computed tomography (CBCT) device, or through reconstruction of 2D images acquired from a fluoroscope, without departing from the scope of the disclosure.

In a further aspect of the disclosure, the fiducials may be automatically identified by an application running on the computer 112. The fiducials may be selected based on the determined pathway to the target. For example, the fiducials may be the bifurcations of the airways that are experienced along the pathway.

Following, the planning phase, where targets are identified and pathways to those targets are created, a navigation phase can be commenced. With respect to the navigation phase, the locating module 110 is employed to detect the position and orientation of a distal portion of the catheter 104. For example, if an EM sensor 205 is employed in catheter 104, the locating module 110 may utilize a transmitter mat 118 to generate an electromagnetic field in which the EM sensor 205 is placed. The EM sensors 205 generate a current when placed in the electromagnetic field is received by the locating module 110 and either five or six degrees of freedom of the position of the sensor 205 and catheter 104 is determined. To accurately reflect the detected position of the catheter 104 in the pre-procedure image data set (e.g., CT or MRI images) or 3D models generated therefrom, a registration process must be undertaken.

Registration of the patient P's location on the transmitter mat 118 may be performed by moving sensor 205 through the airways of the patient P. More specifically, data pertaining to locations of sensor 205, while locatable guide 104 is moving through the airways, is recorded using transmitter mat 118 and locating module 110. A shape resulting from this location data is compared to an interior geometry of passages of the three-dimensional model generated in the planning phase, and a location correlation between the shape and the three-dimensional model based on the comparison is determined, e.g., utilizing the software on computing device 112. In addition, the software identifies non-tissue space (e.g., air filled cavities) in the three-dimensional model. The software aligns, or registers, an image representing a location of sensor 104 with the three-dimensional model and/or two-dimensional images generated from the three-dimension model, which are based on the recorded location data and an assumption that locatable guide 110 remains located in non-tissue space in patient P's airways.

Though described herein with respect to EMN systems using EM sensor 205, the instant disclosure is not so limited and may be used in conjunction with IMU 202, shape sensor 204, optical sensor 206, or ultrasound sensor 210 or without sensors. Additionally, the methods described herein may be used in conjunction with robotic systems such that robotic actuators (not shown) drive the catheter 104 proximate the target.

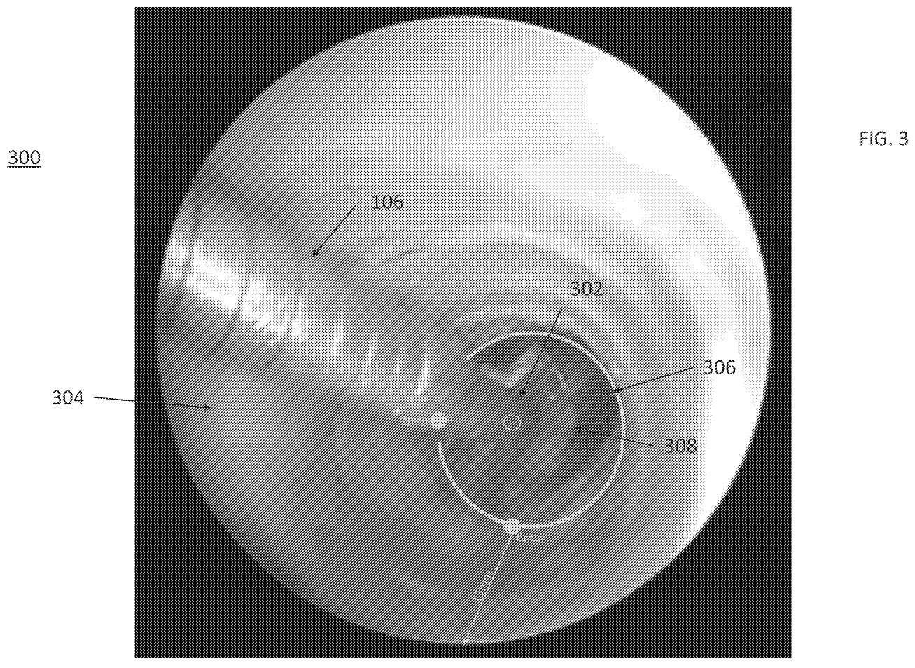

FIG. 3 depicts a live image 300 acquired by the optical sensor 206 as might be displayed in one or more user interfaces of display 114 or monitor 108. The image 300 depicts a tool 106 navigating a luminal network or a patient P. Though not shown in FIG. 3, the tool 106 may include one or more of the sensors described herein above. In one example the tool 106 includes an EM sensor 205. On the live image 300 data regarding the images is also presented. This data may include the distance from the catheter 104, to the distal end 302 of the tool 106. The data may also include a distance of the distal end of the tool 106 from the luminal walls 304. In the image 300 the distance from the catheter 104 to the distal end 302 of the tool 106 is depicted as 15 mm. The distance of the distal end 302 of the tool 106 from one the sidewall of the luminal walls is depicted as 2 mm and from an orthogonal side wall is depicted as 6 mm. A ring 306 may also be displayed on the image depicted where along the luminal wall 304 the distal end 302 of the tool 106 is located. This data provides to the user the depth of view (DOV) of a field of view (FOV) in the image. As a result of this additional data, a clinician may better determine the proximity to a target 308 (e.g., a tumor) which appears in the image 300. Though depicted in image 300 as providing the distances from the distal end 302 of the tool 106 to a left side and a bottom of the luminal wall 304, the depiction of distances may in fact be selected by a user. For example, the application generating the data (described in greater detail below) may depict the closest point of the tool 106 to the luminal wall 304 and one or more other distances. By having two orthogonal distances depicted the relative position of the tool 106 within the lumen can be readily determined by simple comparison of the displayed data and the relative positioning within the lumen. Other data may also be displayed in the image 300. For example, in some instances a target 308 may not be directly discernable in the image generated by the optical sensor 206. Because of the registration process described above, the position of a target, identified in the pre-procedure CT or MRI imaging, may be imported, and displayed in the image 300. Similarly, the pathway to a target may also be displayed in the image 300.

The data displayed on the image 300 may be displayed at any time there is a greater than a predetermined distance between the catheter 104 and the tool 106. Alternatively, the data may be selectively enabled when desired, in this way an overload of data in the image may be eliminated during those times when such data is not necessary, for example when navigating central airways when the target is located in the periphery of the lungs. The data may then automatically begin being displayed when the position of the catheter 104 or tool 106 is within a pre-determined distance to a target. Still further, the display of the data on image 300 may simply be selectively switched on and off as desired by the user.

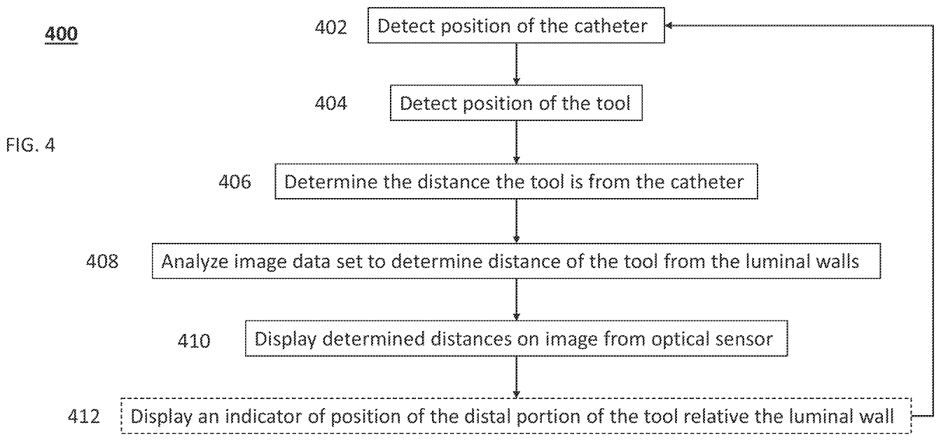

FIG. 4 depicts a method of generating the additional data displayed in image 300. At step 402, the position of the catheter 104 is detected. As described above, this position may be continually being determined by the locating module 110 while the catheter 104 is navigated within the luminal network of the patient during a navigation phase. In a similar fashion the position of the tool 106 may also be detected at step 404. Comparison of the position of the catheter 104 and the tool 106 allows for determination of the distance the tool 106 is from the catheter 104 at step 406.

At step 408, with the position of the tool 106 determined and analysis can be made of an image data set. For example, the pre-procedure CT or MRI image data set which was acquired for planning the navigation to one or more targets. At step 408 the image data set can be analyzed to determine the diameter of the lumen in which the tool 106 is located. In addition, because the position of the tool 106 is known, and the pre-procedure images and 3D models have been registered to the patient, the proximity of the tool's detected position to a luminal wall 304 can also be determined. As noted above, this may be the closest point of the distal portion 302 of the tool 106 to the luminal wall 304 as well as an orthogonal distance, as displayed in FIG. 3.

At step 410, the distances determined in steps 406 and 408 may be displayed on an image acquired by optical sensor 206. The method 400 also provides for an elective step 412 of depicting an indication of the location of the distal portion 302 of the tool 106 on the luminal wall 304. This is depicted in FIG. 3 as the ring 306 on the luminal wall 304. This method 400 may continually update as the tool 106 is advanced further into the luminal network such that the display of the image 300 is updated to depict any change in relative or actual positions of the catheter 104 or tool 106.

Though described in the context of a pre-procedure image data set, the method 400 is not so limited. As noted above, intraprocedural imaging may also be employed to generate data for display in the image 300 acquired by optical sensor 206. For example, cone beam computed tomography (CBCT) or 3D fluoroscopy techniques may be employed as well as other imaging technologies.

Where fluoroscope 116 is employed, the clinician may navigate the catheter 104 and tool 106 proximate a target. Once proximate the target, a fluoroscopic sweep of images may be acquired. This sweep is a series of images (e.g., video) acquired for example from about 15-30 degrees left of the AP position to about 15-30 degrees right of the AP position. Once acquired, the clinician may be required to mark one or more of the catheter 104, tool 106, or target 308 in one or more images. Alternatively, image processing techniques may also be used to automatically identify the catheter 104, tool 106, or target 308. For example, an application running on computer 112 may be employed to identify pixels in the images having relevant Hounsfield units that signify the density of the catheter 104 and tool 106. The last pixels before a transition to a less dense material may be identified as the distal locations of the catheter 104 and tool 106. This may require a determination that the pixels having the Hounsfield unit value indicating a high-density material extent in a longitudinal direction at least some predetermined length. In some instances, the target 308 may also be identified based on its difference in Hounsfield unit value as compared to surrounding tissue. With the catheter 104 and tool 106 positively identified, a 3D volumetric reconstruction of the luminal network can be generated. The 3D volumetric construction may then be analyzed using similar image processing techniques to identify those pixels in the image having a Hounsfield unit signifying the density of the airway wall 304. Alternatively, the imaging processing may seek those pixels having a Hounsfield unit signifying air. In this process, all of the pixels having a density of air are identified until a change in density is detected. By performing this throughout the 3D volumetric construction, the boundaries of the airway wall 304 can be identified. By identifying the airway wall, the diameter of the airway wall can be determined in the areas proximate the catheter 104 or tool 106. Further, the distances the tool 106 is from the airway wall may also be calculated. Accordingly, these additional data, the distance of the distal end 302 of the tool 106 from the catheter 104, the proximity of the tool 106 to the luminal wall 304 and an indicator 306 of the position of the tool relative to the luminal wall 304 can all be depicted on the image 300 generated by the optical sensor 206.

With CBCT, similar processes as those described above with respect to the pre-procedure image data set (i.e., CT or MRI) can be employed. A 3D model may be generated, if desired, depicting the airway. Regardless, image analysis, similar to that described above, can be undertaken to identify the catheter 104 and tool 106. Further, the image processing can determine the diameter of the luminal network in the area proximate the catheter 104 and tool 106. Still further, the position of the catheter 104 and tool 106 within the luminal network can also be identified including the proximity of the catheter 104 or tool 106 to the airway wall. Accordingly, these additional data, the distance of the distal end 302 of the tool 106 from the catheter 104, the proximity of the tool 106 to the luminal wall 304 and an indicator 306 of the position of the tool relative to the luminal wall 304 can all be depicted on the image 300 generated by the optical sensor 206.

In some instances, it may be difficult to determine the position of the tool 106 relative to the catheter 104 using the image processing techniques. Primarily this is because the tool 106 passes through the catheter 104, thus it is difficult to determine where the catheter 104 ends. Accordingly, the sensor data from, for example, the EM sensors 205 located in the catheter 104 and tool 106 may be used to determine the relative position of the catheter 104 and the tool 106 as described above. Thus, the lumen diameter and proximity of the tool 106 to the lumen wall may be determined from the intraprocedure images (CBCT or fluoroscopy) and the distance tool 106 is located relative to the catheter 104 can determined from the sensor data. Accordingly, these additional data, the distance of the distal end 302 of the tool 106 from the catheter 104, the proximity of the tool 106 to the luminal wall 304 and an indicator 306 of the position of the tool relative to the luminal wall 304 can all be depicted on the image 300 generated by the optical sensor 206.

As a result of the processes described hereinabove, the image 300 as depicted in FIG. 3 is provided with additional data detailing the proximity of the tool 106 to the catheter 104. The catheter 104 including a sensor 206 for capturing image 300. The additional data also reveals the relative position of the tool 106 in the lumen in which it is being navigated. As a result of this additional data the FOV in the image 300 is numerically afforded a DOV. Clinicians receiving this additional data are thus provided similar context for the image 300 to that achieved when stereoscopic imaging is undertaken. However, the methods and systems described here require only the use of a single optical sensor at the end of the catheter to achieve this context for the clinician.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments.

Throughout this description, the term “proximal” refers to the portion of the device or component thereof that is closer to the clinician and the term “distal” refers to the portion of the device or component thereof that is farther from the clinician. Additionally, in the drawings and in the description above, terms such as front, rear, upper, lower, top, bottom, and similar directional terms are used simply for convenience of description and are not intended to limit the disclosure. In the description hereinabove, well-known functions or constructions are not described in detail to avoid obscuring the disclosure in unnecessary detail.

What is claimed is:

1. A system for depicting a depth of view (DOV) in an image comprising:
a catheter including a first sensor configured for navigation in a luminal network and an optical sensor for generating an image;
a tool including a second sensor configured to pass through a working channel in the catheter;
a locating module configured to detect a position of the catheter and the tool; and
an application stored on a computer readable memory and configured, when executed by a processor to execute the steps of:
registering data received from the first or second sensor with an image data set;
analyzing an image data set to determine a diameter of a luminal network proximate the second sensor; and
displaying the image generated by the optical sensor in combination with an indicator of a relative position of the catheter and tool and an indicator of a position of a distal portion of the tool relative to a luminal wall of the luminal network.

2. The system of claim 1, wherein the application executes a step of displaying distance of a closest point of the distal portion of the tool relative to the luminal wall.

3. The system of claim 1, wherein the application executes a step of displaying at least two orthogonally measured distances of the distal portion of the tool relative to the luminal wall.

4. The system of claim 1, wherein the image data set is a pre-procedure image data set.

5. The system of claim 1, wherein the image data set is an intraprocedure image data set.

6. The system of claim 5, wherein the intraprocedure image data set is received from a fluoroscope.

7. The system of claim 1, wherein the sensor located in the catheter and in the tool are electromagnetic sensors.

8. The system of claim 1, wherein the sensor located in the catheter and in the tool are inertial measurement units.

9. The system of claim 1, wherein the sensor located in the catheter and in the tool are shape sensors.

10. The system of claim 1, wherein the sensor located in the catheter and in the tool are ultrasound sensors.

11. A method of assessing a depth of view of an image comprising:
determining a position of a catheter in a luminal network;
determining a position of a tool relative the catheter in the luminal network;
acquiring an image data set;
analyzing the image data set to determine a diameter of the luminal network proximate the determined position of the tool;
displaying an image acquired by an optical sensor secured to the catheter; and
displaying an indicator of a relative position of the catheter and tool in the image acquired by the optical sensor and an indicator of a position of a distal portion of the tool relative to a luminal wall of the luminal network.

12. The method of claim 11, further comprising displaying a distance of a closest point of the distal portion of the tool relative to the luminal wall.

13. The method of claim 12, wherein the indicator includes at least two orthogonally measured distances of the distal portion of the tool relative to the luminal wall.

14. The method of claim 11, wherein the image data set is a pre-procedure image data set.

15. The method of claim 11, wherein the image data set is an intraprocedural image data set.

16. The method of claim 11, wherein the position of the catheter is determined from data received from a sensor located in the catheter.

17. The method of claim 16, wherein the position of the tool is determined from data received from a sensor located in the tool.

18. The method of claim 17, wherein the sensor located in the catheter and in the tool are electromagnetic sensors.

19. The method of claim 17, wherein the sensor located in the catheter and in the tool are inertial measurement units.

20. The method of claim 17, wherein the sensor located in the catheter and in the tool are shape sensors.
